(19) United States
(12) Patent Application Publication
(10) Pub. No.: US 2008/0154117 A1
(43) Pub. Date: Jun. 26, 2008

(54) MAGNETIC RESONANCE METHOD AND APPARATUS FOR ACQUISITION OF IMAGE DATA OF A VESSEL WALL

(76) Inventor: Sonia Nielles-Vallespin, Nurnberg (DE)

Correspondence Address: SCHIFF HARDIN, LLP;PATENT DEPARTMENT, 6600 SEARS TOWER, CHICAGO, IL, 60606-6473, US

(21) Appl. No.: 11/954,016

(22) Filed: Dec. 11, 2007

(30) Foreign Application Priority Data
Dec. 11, 2006 (DE) 10 2006 058 316.7

(51) Int. Cl. A61B 5/055

(52) U.S. Cl. 600/410

(57) ABSTRACT

In a magnetic resonance method and apparatus for acquisition of an image for examination of a vessel wall variation, a vessel wall section of a patient to be examined is positioned in an imaging volume of the magnetic resonance apparatus, image data of the vessel wall section are acquired with an ultrashort echo time sequence, and an image is generated from the acquired image data.

### BACKGROUND OF THE INVENTION

1. Field of the Invention

The present invention concerns a method for acquisition of image data of a vessel wall by means of magnetic resonance technology as is used for diagnosis of variations of the vessel wall that are due to an atherosclerosis. The invention also concerns a magnetic resonance apparatus for implementing such a method.

2. Description of the Prior Art

Arteriosclerosis is a systemic illness of the arteries that leads to deposits of blood lipids, thromboses, connective tissue and calcium in the vessel walls. The focal variations that occur in the inner and in the middle vessel wall are also referred to as atherosclerosis. The atherosclerotic variations are often locally limited and form what are known as plaques. Among other things, heart infarcts and strokes are among the typical results of arteriosclerosis.

Thromboembolic events, i.e. the formation of a blood clot in an artery, are often based on a rupture of a “vulnerable” plaque, namely the tearing of the thin fibrous cap of the inflamed vessel wall variation.

The vulnerability of a plaque appears to be substantially more significantly influenced by the tissue composition of the plaque than by the size of the plaque and the remaining size of the vessel lumen. Primarily calcium deposits (calcified tissue), connective tissue, lipid deposits and fibrin deposits are among the tissue components of a plaque.

A number of methods exist in order to be able to examine a vessel wall variation.

Intravascular ultrasound (IVUS) allows a radiation-free examination of the vessel wall, and is predominantly suitable for soft, non-calcified plaque, but is an invasive examination method and is relatively expensive.

Examination methods based on computed tomography entail a relatively high radiation exposure for the patient to be examined.

Magnetic resonance (MR) technology is also used for diagnosis of arteriosclerosis. The MR technique is a technique known for some decades with which images of the inside of an examination subject can be generated. Greatly simplified, to generate an MR image the examination subject is positioned in a strong, static, homogeneous basic magnetic field (field strengths of 0.2 Tesla to 7 Tesla and more) in an MR apparatus so the nuclear spins thereof orient along the basic magnetic field. Radio-frequency excitation pulses are radiated into the examination subject to excite nuclear magnetic resonances, the triggered nuclear magnetic resonances being measured (deleted) and MR images being reconstructed therefrom. For spatial coding of the measurement data, rapidly switched magnetic gradient fields are superimposed on the basic magnetic field. The acquired measurement data are digitized and stored in a mathematical organization called a k-space matrix as complex numerical values. By multi-dimensional Fourier transformation, an MR image can be reconstructed from the data in the k-space matrix. The temporal series of the excitation pulses and the gradient fields for excitation of the image volume to be measured, for signal generation and for spatial coding is designated as a sequence (or also as a pulse sequence or measurement sequence).

The MR technique is also used for angiography by the application of specific sequences. MR angiography is used for examination of the lumen of a vessel and thus for detection of a possibly present stenosis. The size of the lumen, however, does not correlate with the vulnerability of a plaque to rupture, which is why at-risk patients can be only insufficiently identified with this examination method.

One possibility to be able to quantify atherosclerotic vessel variations is described in the document by J. M. A. Hofman et al., “Quantification of Atherosclerotic Plaque Components Using In Vivo MRI and Supervised Classifiers”, Magn. Res. Med. 55(4), 790-799, 2006. Various T1-weighted, T2-weighted and proton density-weighted sequences are used for image acquisition of atherosclerotic vessel wall variations. Further analysis of this approach has shown that calcifications and/or calcium deposits in a plaque can be only insufficiently detected since calcium, due to its short T2 relaxation time, appears in the image as a region with signal attenuation. Signal attenuations, however, can also be based on various artifacts, such that calcifications are often overestimated.

A sequence known as an ultrashort echo time sequence (UTE sequence in the following) with which signals of tissue components with a short T2 relaxation time can be measured before the transverse magnetization has decayed, is disclosed in WP 2005/026748 and in the article by P. D. Gatehouse and G. M. Bydder, “Magnetic Resonance Imaging of Short T2 components in Tissue”, Clin Radiol 58(1), 1-19, 2003.

### SUMMARY OF THE INVENTION

An object of the present invention is to provide a method for acquisition of an image of a vessel wall that enables a non-invasive, x-ray-free and high-resolution image acquisition with which an image of an atherosclerotic vessel wall variation can be acquired. The method should allow an improved evaluation of the composition of the vessel wall variation and an improved identification of patients at risk for a thromboembolic event. Furthermore, it is an object of the invention to provide a magnetic resonance apparatus for implementation of such a method.

The above object is inventively achieved by a method for acquisition of an image for examination of a vessel wall variation according to the invention, including positioning a vessel wall section of a patient to be examined in an imaging volume of a magnetic resonance apparatus, acquisition of image data of the vessel wall section with an ultrashort echo time sequence, and generating an image from the acquired image data.

A suitable ultrashort echo time sequence is described in WO 2005/026748 and in the document by P. D. Gatehouse and G. M. Bydder, “Magnetic resonance Imaging of Short T2 Components in Tissue”, Clin Radiol 58(1), 1-19, 2003. A UTE sequence is characterized by an echo time TE of less than 100 μs (microseconds), advantageously less than 80 μs.

The imaging with an ultrashort echo time sequence is based on a short, advantageously non-selective RF excitation pulse with subsequent acquisition of signals from excited nuclear spins. In order to enable the desired short echo times, the acquisition of the measurement data already ensues during the ramp phase while the gradient fields switched for acquisition of the measurement data are being established.

In a three-dimensional ultrashort echo time sequence, for example, gradient fields are radiated that enable an asymmetrical acquisition of the measurement data from the center of k-space radially outwardly—for example to a surface of a sphere in k-space.

It is possible to also measure signals of tissue components with a short T2 relaxation time (such as, for example, calcified tissue) so that this tissue also generates a positive contrast (i.e. a visible signal) in the image. In the generated image this is advantageous since now calcifications (which generate only a negative contrast with conventional MR sequences, i.e. generate only an insufficient signal in the representation) can be made visible. The generated image allows a user to better assess the composition of a vessel wall variation. Computer-aided evaluation algorithms based on the generated image data can likewise implement a more precise quantification of tissue components of a plaque since now one of the components that is essential for a diagnosis of the vulnerability of a plaque, namely calcifications or calcium deposits, generates a distinctly visible and measurable signal.

In an embodiment, the ultrashort echo time sequence includes at least one radio-frequency saturation pulse for suppression of signals of nuclear spins of fat tissue. In this embodiment, it is possible to reduce signals that have their origin in nuclear spins of fat tissue since these nuclear spins are saturated by the radio-frequency saturation pulse. The contrast between lipid deposits and calcifications in a vessel wall hereby increases.

In another embodiment, the ultrashort echo time sequence includes at least one radio-frequency saturation pulse for suppression of signals of nuclear spins whose T2 relaxation time is greater than a predetermined threshold. It is thereby possible to reduce signals that have their origin in tissue with a long T2 relaxation time. In the generated image this causes a higher contrast between this type of tissue and calcified tissue.

K-space is advantageously three-dimensionally scanned with the ultrashort echo time sequence. The scanning of k-space preferably ensues in a radial manner. Such a scanning trajectory shows a relatively low susceptibility to movement artifacts and additionally allows the production of an image with a small image region FOV (field of view) with high resolution.

The ultrashort echo time sequence is advantageously triggered by an acquired navigator signal. With the use of the navigator signal it is possible to detect various movements of the body (for example breathing movements) and to match the acquisition of the measurement data to these.

The above object also is achieved in accordance with the present invention by a magnetic resonance apparatus that is configured to implement the above-described method and all embodiments thereof.

### BRIEF DESCRIPTION OF THE DRAWINGS

FIG. 1 is a schematic illustration of the basic components of an MR apparatus.

FIG. 2 shows the method steps of an embodiment of the invention.

FIG. 3 schematically illustrates a three-dimensional UTE sequence.

FIG. 4 schematically illustrates a three-dimensional multi-echo UTE sequence.

FIG. 5 schematically illustrates a UTE sequence that is triggered by an ECG signal and a navigator signal.

### DESCRIPTION OF THE PREFERRED EMBODIMENTS

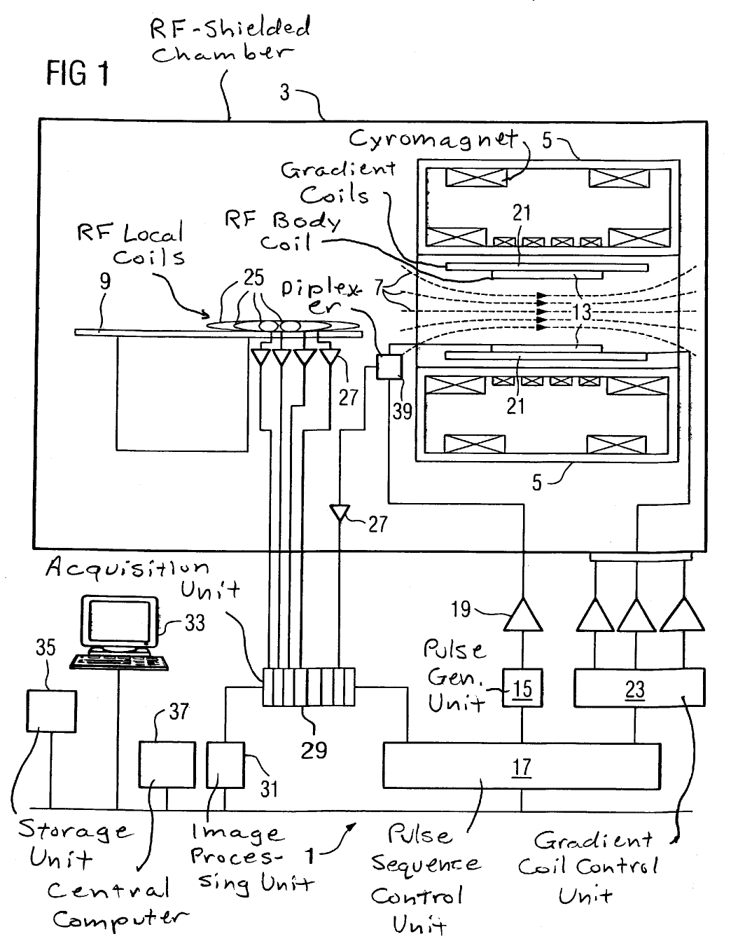

FIG. 1 schematically shows the basic design of a magnetic resonance apparatus 1. In order to examine a body by means of magnetic resonance imaging, various magnetic fields matched as precisely as possible to one another in terms of their temporal and spatial characteristics are applied.

A strong magnet (typically a cryomagnet 5 with a tunnel-shaped opening) arranged in a radio-frequency-shielded measurement chamber 3 generates a static, strong basic magnetic field 7 that is typically 0.2 Tesla to 3 Tesla and more. A body or a body part (not shown) to be examined is surprised on a patient bed 9 and positioned in a homogeneous region of the basic magnetic field 7.

The excitation of the nuclear spins of the body ensues by radio-frequency excitation pulses that are radiated by a radio-frequency antenna (shown here as a body coil 13). The radio-frequency excitation pulses are generated by a pulse generation unit 15 that is controlled by a pulse sequence control unit 17. After amplification by a radio-frequency amplifier 19 they are conducted to the radio-frequency antenna. The radio-frequency system shown here is only schematically indicated. Typically more than one pulse generation unit 15, more than one radio-frequency amplifier 19 and a number of radio-frequency antennas are used in a magnetic resonance apparatus 1.

Furthermore, the magnetic resonance apparatus 1 has gradient coils 21 with which gradient fields for selective slice excitation and for spatial coding of the measurement signal are radiated in a measurement. The gradient coils 21 are controlled by a gradient coil control unit 23 that, like the pulse generation unit 15, is connected with the pulse sequence control unit 17.

The signals emitted by the excited nuclear spins are acquired by the body coil 13 and/or by local coils 25 are amplified by associated radio-frequency preamplifiers 27, and are further processed and digitized by an acquisition unit 29.

Given a coil (such as, for example, the body coil 13) that can be operated both in transmission mode and in acquisition mode, the correct signal relaying is regulated by an upstream transmission-reception diplexer 39.

An image processing unit 31 generates from the measurement data an image that is presented to a user via an operating console 33, or is stored in a storage unit 35. A central computer 37 controls the individual system components. The computer 37 of the magnetic resonance apparatus 1 is fashioned such that a method according to the invention can be implemented with the magnetic resonance apparatus 1.

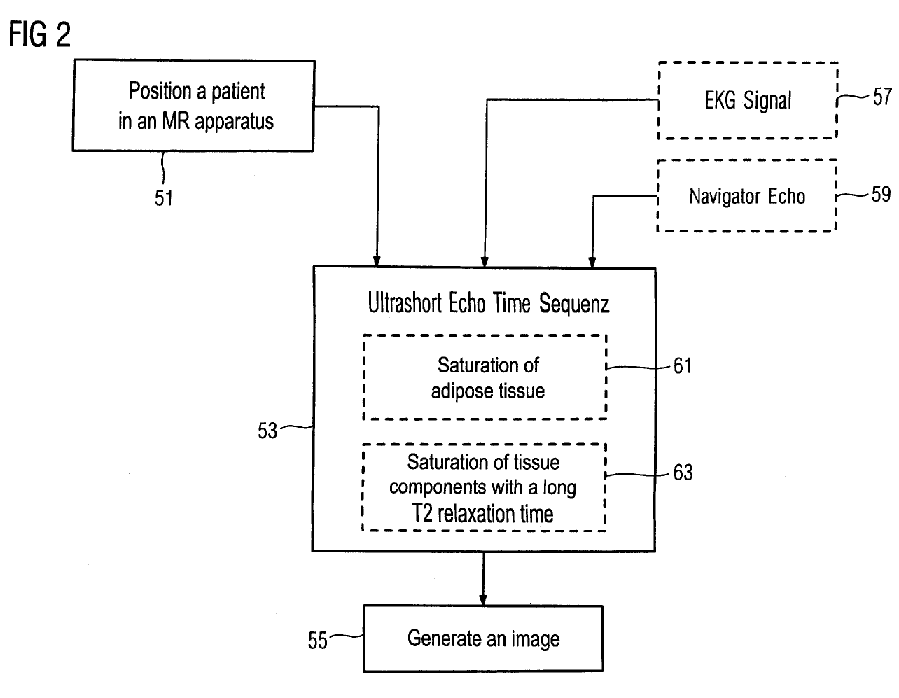

FIG. 2 shows an overview of the method steps of an advantageous embodiment of the inventive method.

In a first step 51 a patient is positioned in an imaging volume of a magnetic resonance apparatus such that an image of a vessel section to be examined can be acquired.

In a second step 53, an image of the vessel section to be examined is produced with a UTE sequence. A UTE sequence is characterized in that with it those tissues with a very short T2 relaxation time (for example with a relaxation time of under 10 ms such as, for example, calciferous tissue) being also clearly visible in the image.

In a third step 55, the image of the vessel section is generated. A user can thereupon visually assess the image or also effect further evaluations (manually and/or automatically) on the image, for example for quantification of the individual tissue components. Even calcifications can now be detected more precisely.

Additional optional steps advantageously augment the method.

An ECG signal or a navigator echo of the patient can be acquired in a fourth step 57 and fifth step 59 for prospective data acquisition correction. Both are used for triggering the data acquisition since movement artifacts (as can arise, for example, from the movement of the beating heart or from breathing movements) can thereby be distinctly reduced.

The UTE sequence also can be augmented so as to saturate nuclear spins of fat tissue 61 or to saturate nuclear spins with a long T2 relaxation time 63, for example with a T2 relaxation time that lies above a predefined threshold. For example, this occurs by the UTE sequence including a suitably fashioned radio-frequency saturation pulse. The contrast difference from calcifications to fat tissue or to other tissue components can be increased in this manner.

It is also possible, for example, to use a double echo sequence in which two signal echoes with different echo times T_{E1} and T_{E2} are acquired after an excitation pulse. Suppression of nuclear spins with a long T2 relaxation time can ensue by subtracting the signal echo with the long echo time T_{E2} is from the signal echo with the short echo time T_{E1}.

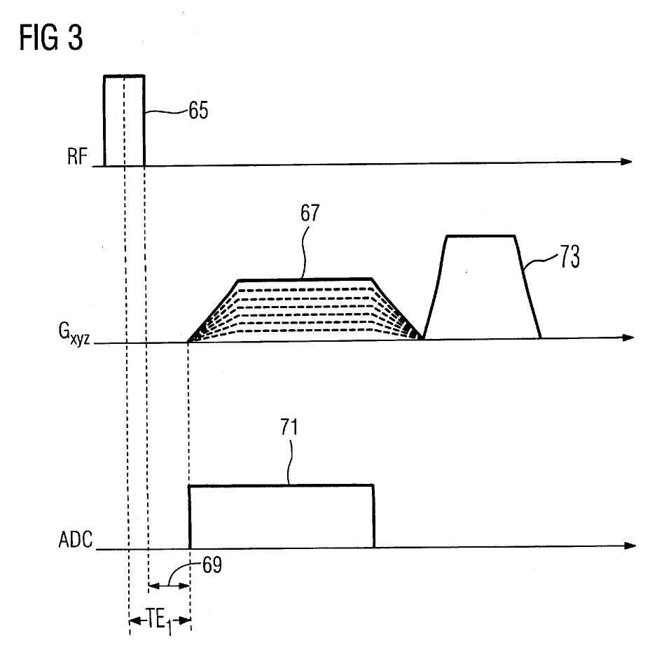

FIG. 3 shows a schematic representation of a three-dimensional UTE sequence. The first line RF shows a radiated radio-frequency excitation pulse 65 for non-selective excitation of nuclear spins. The second line G_{xys} schematically shows the gradient fields that are applied in the x-direction, y-direction and z-direction.

The application of readout gradient fields 67 generates a gradient echo that is scanned after a delay time 69 following the radio-frequency excitation pulse 65 (third line ADC for “analog to digital conversion”). The scanning 71 ensues at a point in time TE₁ at which a measurable signal from tissues with a short T2 relaxation time (such as, for example, calcified tissue) is also still present. In order to achieve short echo times on the order of multiples of 10 μs, the acquisition of the measurement data already ensues at the point in time at which the readout gradient fields 67 are still located in the ramp phase. After acquisition of the measurement data, a spoiler gradient 73 destroys a possibly still present transverse magnetization before a new excitation pulse.

The scanning of k-space thereby ensues radially from the center of k-space outward. This scanning corresponds to a scanning along a k-space ray that, beginning from the center, points toward the surface of a sphere or an ellipsoid. In order to achieve a homogeneous distribution of the measurement data in k-space, various known algorithms can be applied with which a number N of different k-space rays are optimally homogeneously distributed in k-space.

The direction of a k-space ray can thereby be characterized by two spatial angles, namely by the polar angle θ (0<θ<π) and the azimuthal angle φ (0<φ<2π). Given a predetermined direction of a k-space ray, the gradients Gₓ, G_{y} and G_{z} in the x-direction, y-direction and z-direction can be calculated as follows:

G_{X}=G sin θ cos φ

G_{Y}=G sin θ sin φ

G_{Z}=G cos θ

This radial three-dimensional k-space scanning provides a number of advantages. This scanning is relatively insensitive to movement artifacts such despite the movement an image with only slight artifacts can be acquired even in the case of pulsing vessels. Moreover, this scanning also allows the representation of small image regions (FOV) with a high resolution, which is important for the presentation of atherosclerotic vessel wall variations. This scanning additionally allows scanning of the image region with an isotropic resolution, which improves the imaging of the vessel.

Although advantageous, a three-dimensional scanning of k-space is not strictly necessary. Two-dimensional UTE sequences can also be applied.

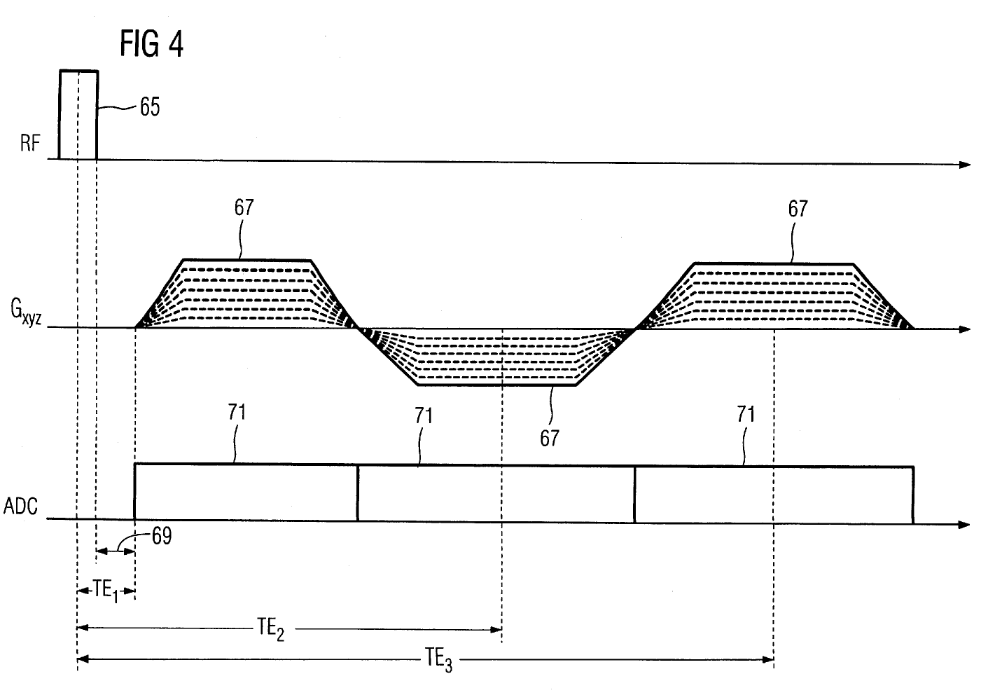

FIG. 4 shows a schematic representation of a three-dimensional UTE sequence that is fashioned as a multi-echo sequence.

In comparison to the sequence shown in FIG. 3, readout gradient fields 67 are applied repeatedly and respectively generate a gradient echo that is read out at different points in time (TE₁, TE₂, TE₃). In this manner different images that respectively exhibit a different contrast can be generated with only one sequence. These images can be combined with one another in various ways.

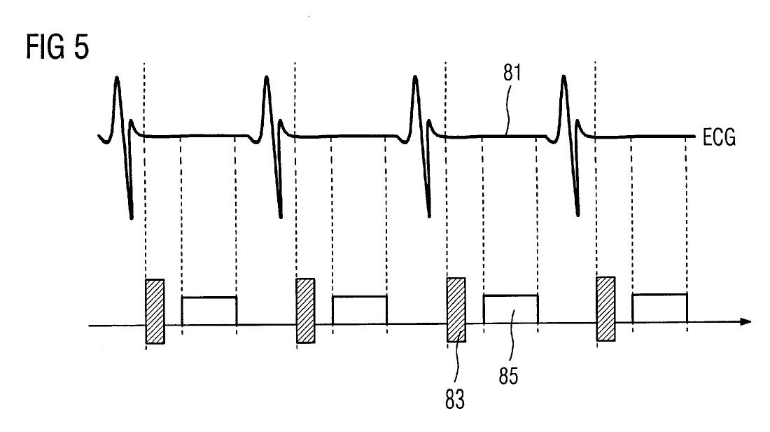

FIG. 5 schematically shows the temporal course of a UTE sequence whose data acquisition segments 61 are triggered by an acquired electrocardiography (ECG) signal 57 and by an acquired navigator signal 59. These triggerings offer the advantage to adapt the UTE sequence in terms of its temporal and spatial characteristics such that movements of the heart and the lungs can be compensated in an advantageous manner. This is particularly advantageous in the imaging of coronary arteries. The movements caused by the heart and by the lungs thereby lead only to a slight reduction of the image quality.

Although modifications and changes may be suggested by those skilled in the art, it is the intention of the inventor to embody within the patent warranted hereon all changes and modifications as reasonably and properly come within the scope of her contribution to the art.

I claim as my invention:

1. A method for acquiring an image of an in vivo vessel wall variation, comprising the steps of:
positioning a vessel wall section in a patient in an imaging volume of a magnetic resonance apparatus;
acquiring magnetic resonance image data of the vessel wall section using an ultrashort echo time sequence; and
reconstructing an image of said vessel wall section from the acquired magnetic resonance image data.

2. A method as claimed in claim 1 comprising, as said ultrashort echo time sequence, using an ultrashort echo time sequence having an echo time T_{E} that is less than 100 μs.

3. A method as claimed in claim 1 comprising, as said ultrashort echo time sequence, using an ultrashort echo time sequence comprising at least one radio-frequency saturation pulse that suppresses signals of nuclear spins of fat tissue.

4. A method as claimed in claim 1 comprising, as said ultrashort echo time sequence, using an ultrashort echo time sequence comprising at least one radio-frequency saturation pulse that suppresses signals of nuclear spins having a T2 relaxation time that is greater than a predetermined threshold.

5. A method as claimed in claim 1 comprising entering said raw magnetic resonance data into k-space by three-dimensionally scanning k-space with said ultrashort echo time sequence.

6. A method as claimed in claim 5 comprising radially scanning k-space.

7. A method as claimed in claim 1 comprising obtaining an ECG signal from the patient and triggering said ultrashort echo time sequence with said ECG signal.

8. A method as claimed in claim 1 comprising obtaining a navigator signal and triggering said ultrashort echo time sequence with said navigator signal.

9. A magnetic resonance apparatus comprising:
a data acquisition unit configured to receive a patient therein, said patient comprising an in vivo vessel wall section exhibiting a vessel wall variation;
a control computer that operates said data acquisition unit to acquire raw magnetic resonance data from the patient in the data acquisition unit with an ultrashort echo time sequence; and
an image reconstruction computer that reconstructs an image of the vessel wall section, in which said vessel wall variation is visible, from the acquired magnetic resonance raw data.
